(19) United States
(12) Patent Application Publication
(10) Pub. No.: US 2003/0086527 A1
(43) Pub. Date: May 8, 2003

(54) Radiation modulating apparatus and methods therefore

(76) Inventors: Burton L. Speiser, Paradise Valley, AZ (US); Erdal M. Gurgoze, Cave Creek, AZ (US); Kevin L. Rogers, Phoenix, AZ (US)

Correspondence Address: Snell & Wilmer, One Arizona Center, 400 E. Van Buren Street, Phoenix, AZ, 85004-2202, US

(21) Appl. No.: 10/001,024

(22) Filed: Nov. 2, 2001

(51) Int. Cl. A61N 5/10

(52) U.S. Cl. 378/65

(57) ABSTRACT

A radiation modulating device and methods enhance the delivery of radiation by modulating the intensity of a radiation beam. A radiation modulating device may be placed between any radiation source and any target such that at least some portion of the radiation beam emitted from the source passes through the radiation modulating device before reaching a target. In one embodiment of the present invention, a radiation modulating device may include a housing and a core. A housing may be configured to support a core and a core may be configured to partially or wholly block one or more portions of radiation emitted from a radiation source. The configuration of the core may be based on a distribution of intensities of radiation desired to be delivered to a target. Though a radiation modulating device according to various aspects of the present invention may be used for any of a number of purposes, one use for the device is to enhance the delivery of radiation therapy to cancer patients.

### FIELD OF THE INVENTION

The present invention relates generally to the field of radiation devices. More specifically, the present invention relates to new apparatus and methods that can be used in a variety of manners, for example in enhancing the delivery of radiation.

### BACKGROUND OF THE INVENTION

Cancer is the second leading cause of death in the United States, exceeded only by heart disease. The American Cancer Society predicted that 1,220,100 new cancer cases would be diagnosed in the U.S. in the year 2000 and that 552,200 Americans would die of the disease. At that rate, 1,500 Americans die each day of cancer and one in four deaths are caused by cancer. In the 1990's alone, approximately 13 million new cases of cancer were diagnosed, not including noninvasive cancers or basal and squamous cell skin cancers.

The National Institutes of Health estimated that the overall financial costs for cancer in the year 2000 would reach $180.2 billion. Of that total, $60 billion were for direct medical costs (total of all health expenditures), 15 billion were for indirect morbidity costs (cost of lost productivity due to illness), and $105.2 billion were for indirect mortality cost (cost of lost productivity due to premature death). Thus, cancer adds significantly to both the death tolls and the national health care costs of the United States. Similar statistics for other countries demonstrate that cancer is an equally serious health problem throughout the world.

Typically, three medical techniques are used to combat cancer—surgery, chemotherapy and radiation therapy. These techniques are often used in combination and each has advantages and disadvantages. In radiation therapy, used in treatment of nearly two-thirds of all cancer patients, high-energy rays are used to damage cancer cells and stop them from growing and dividing. Thus, radiation therapy may stop the growth of a cancerous tumor and even destroy all the cells of the tumor. Where complete tumor destruction is not possible, radiation may be used to shrink a tumor, so that it can be more easily removed surgically. Radiation may also be used after surgery to destroy microscopic remnants of the cancer which were not removed during surgery.

Like surgery, radiation therapy is designed to be a local treatment, affecting cancer cells only in the treated area. This is in contrast to chemotherapy, which kills cancer cells throughout the body, as well as many healthy cells. While the goal of radiation therapy is to deliver radiation only to the treatment area, various limitations exist. Radiation can come from a machine (external radiation) or an implant (a small container of radioactive material) placed into or near the tumor (internal radiation). In either case, radiation inevitably passes through healthy, normal parts of the patient's body which surround the cancer, often damaging normal structures. For example, in treating prostate cancer with external radiation therapy, the radiation beam may pass through portions of the skin, rectum, bladder and genitalia, often causing inflammation and potentially serious damage to those tissues and organs.

To minimize these negative effects, it is important to deliver as much radiation to a target area as possible, while minimizing the amount of radiation delivered to surrounding, healthy structures. Advances in radiation therapy techniques aid in this regard. One such technique is called “intensity modulated radiation therapy” (“IMRT”). As with most radiation therapy techniques, IMRT involves two stages—treatment planning and radiation delivery. Treatment planning involves examining the area of cancer in a patient via imaging studies, such as computed tomography studies (“CT scans”) and/or magnetic resonance imaging (“MRI”). Typically, a clinician responsible for treatment planning, such as a radiation oncologist, then defines the optimal dose of radiation to be delivered to the treatment site and the tolerable level of radiation to be administered to the surrounding tissues. Sophisticated computer software processes the imaging information and clinician-defined parameters to create a treatment plan. Another set of sophisticated software then translates the treatment plan into instructions for a radiation delivery device. A radiation delivery device used for IMRT typically includes a linear accelerator, which creates a beam of radiation, and a radiation beam collimator (or multi-leaf collimator “MLC”), which blocks portions of the beam for specified time intervals. Both the accelerator and the MLC are typically controlled by instructions formulated by the computer software mentioned above.

The objective of IMRT is to partially block portions of a radiation beam that pass through important healthy bodily structures, to reduce radiation doses to those structures, while allowing as much of the radiation beam as possible to arrive at a cancerous target area. Treatment results have shown that this objective is being met. For example, IMRT has been used successfully over the past several years to treat prostate cancer while decreasing doses to the rectum and bladder, at such hospitals as Memorial Sloan Kettering Cancer Center.

While IMRT techniques provide a potentially revolutionary approach to radiation therapy, the vast majority of the clinics, hospitals and other facilities that provide radiation therapy cannot afford the equipment necessary to provide it. As healthcare costs increase throughout the world, healthcare providers must make difficult choices regarding how to provide the best possible services at prices their patients can afford. Although most clinicians would prefer to purchase every cutting edge medical technology available, they simply cannot afford to do so while still caring for their patients in an affordable manner.

The basic machinery needed to provide IMRT is a linear accelerator, an MLC, a computer system for controlling the accelerator and the MLC, and a computer system for creating treatment plans. Unfortunately, only about 10% of the currently used radiation therapy systems can be upgraded with these components to provide IMRT. These currently used systems are typically incompatible because linear accelerators must be digital to be upgraded to provide IMRT, and most currently available systems use analog accelerators. A new IMRT radiation therapy system typically costs between $1,300,000 and $2,500,000.

Upgrading a digital radiation therapy system to provide IMRT is also a costly undertaking. Generally, upgrading requires purchasing an MLC, which may cost between $150,000 and $600,000, additional system upgrades (e.g., to the linear accelerator) that may cost between $200,000 and $500,000, and a treatment planning system that may cost between $180,000 and $300,000. Thus, total costs for upgrading a compatible digital accelerator typically run from $530,000 to $1,400,000.

In addition to the high costs of switching from conventional radiation therapy systems to IMRT systems, currently available systems for providing IMRT have further disadvantages. For example, such systems often require longer treatment times, typically resulting in increased patient movement during treatments and, thus, less precise treatments. Currently available IMRT systems also usually require more expensive and complex maintenance than conventional systems.

Therefore, a need exists for a device and methods that enable conventional radiation therapy systems to be adapted to provide innovative radiation therapy techniques in a cost effective manner.

### SUMMARY OF THE INVENTION

The present invention addresses the aforementioned need by providing a radiation modulating device and methods for making and using same. In one embodiment, a radiation modulating device may be used to enhance the delivery of radiation by existing radiation delivery systems so that sufficient radiation is delivered to a target and limited radiation is delivered to areas surrounding the target. For example, in accordance with one aspect of this embodiment of the present invention, a radiation modulating device is provided, at least one section of which is configured for wholly or partially blocking one or more portions of a radiation beam. A radiation beam blocked in such a manner will have varying intensities across its field when it reaches a target. Thus, a radiation modulating device may configured to specifically vary the intensities of a radiation beam to have a given effect on a target and/or on areas surrounding a target. For example, a radiation modulating device may be configured to allow a sufficient intensity of a radiation beam to be delivered to one area of a cancerous tumor, while limiting the intensity of the beam being delivered to a vital structure surrounding the tumor.

In accordance with one aspect of the invention, a method for producing a radiation modulating device involves first collecting information. This information may include information about a target, such as imaging data, treatment parameters and the like. Information may also include details about how a radiation modulating device is to be used, such as what type of system it will be used with, where it will be placed within the system and the like. Once information is collected, it may used to create a radiation delivery device. For example, information may be input into a computer or similar system which may use the information as instructions for one or more machines configured to construct a radiation modulating device. The construction process may be accomplished in any suitable manner, such as milling of a solid piece of material, milling a mold which can be used to build a radiation modulating device, adhering multiple pieces of material together to make a device, or any other suitable process.

Once built, a radiation modulating device may be used for any number of functions. In one embodiment, a radiation modulating device may be used to modulate the intensity of a radiation beam used in radiation therapy of a cancer patient.

### BRIEF DESCRIPTION OF THE DRAWINGS

Additional aspects of the present invention will become evident upon reviewing the non-limiting embodiments described in the following specification and claims taken in conjunction with the accompanying drawing figures, wherein like numerals designate like elements, and:

FIG. 1 is partial, cut-away, perspective view of a device according to one embodiment of the present invention;

FIG. 2a is a complete top view of a device such as shown in FIG. 1, with a dotted line and shaded region designating the section cut away in FIG. 1.

FIG. 2b is a complete side view of a device such as shown in FIG. 1, with a dotted line and shaded region designating the section cut away in FIG. 1.

FIG. 3 is a flow chart showing a method for manufacturing a device according to one embodiment of the present invention;

FIG. 4 is a flow chart showing various aspects of a method for manufacturing a device according to one embodiment of the present invention;

FIG. 5 is an example of a radiation beam intensity map, used as part of a method for manufacturing a device according to one embodiment of the present invention;

FIG. 6 is an example of a modulating device thickness map, used as part of a method for manufacturing a device according to one embodiment of the present invention;

FIG. 7a is copy of a radiograph developed by a radiation beam delivered by a radiation therapy system including a device according to one embodiment of the present invention;

FIG. 7b is a copy of a radiograph developed by a radiation beam delivered by a currently available IMRT system;

FIG. 8 is a graph of radiation doses delivered by a currently available IMRT system and a system including a device according to one embodiment of the present invention, the radiation doses being measured at a target.

### DETAILED DESCRIPTION

The following description is of exemplary embodiments only and is not intended to limit the scope, applicability, or configuration of the invention in any way. Rather, the following description merely provides convenient illustrations for implementing exemplary embodiments of the invention. For example, various changes may be made in exemplary embodiments, without departing from the scope of the invention as set forth in the appended claims. Additionally, the following description often describes embodiments of the present invention in the context of providing radiation therapy to cancer patients and often refers to users of the present invention as “clinicians.” It should be understood that radiation therapy of cancer patients is merely an exemplary context in which various embodiments of the invention may be used. Furthermore, various embodiments of the invention may be used by any suitable users. For the purposes of this application, therefore, “clinicians” means radiation oncologists, dosimetrists, radiation physicists or any other person generally suitable to use any of the various aspects of one or more exemplary embodiments of the invention.

Generally, in accordance with one embodiment of the present invention, a radiation modulating device is provided which is configured to enhance radiation delivery. Such a device may generally be placed between a radiation source and a target. In one embodiment of the invention, a radiation modulating device is configured such that at least one section of the device, when placed between a radiation source and a target, will block one or more portions of a radiation beam to a greater extent than one or more other portions of the beam. Thus, according to one aspect of the present invention, a radiation modulating device may cause a radiation beam, which has approximately equal intensities across its field when it leaves a radiation source, to have different intensities across its field when it reaches a target.

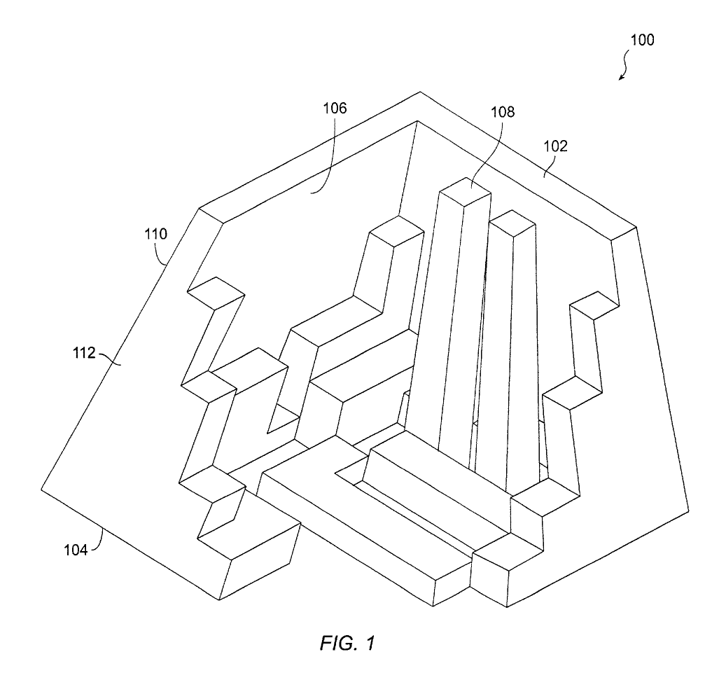

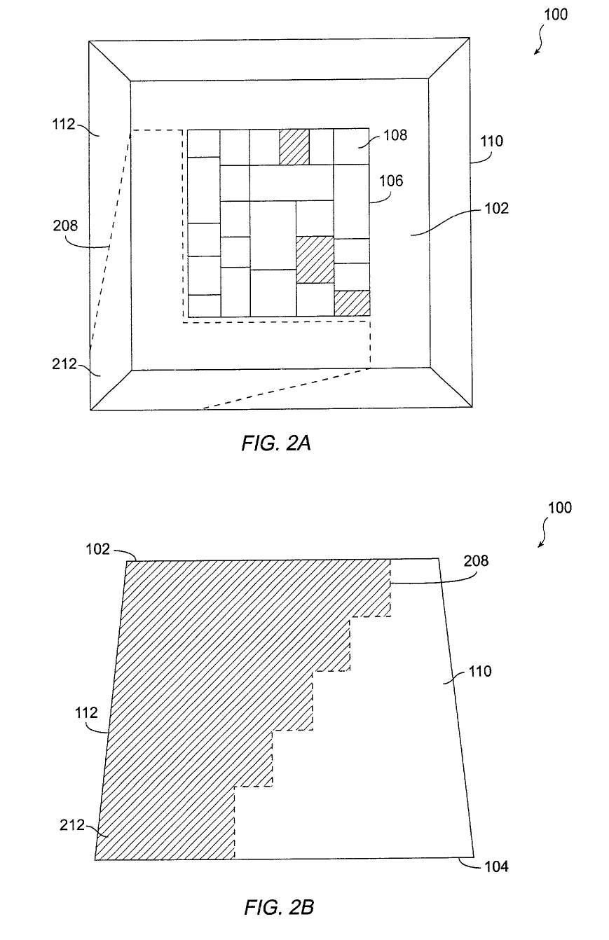

In one embodiment of the present invention, and with reference to FIGS. 1, 2a and 2b, a radiation modulating device 100 may take the form of a block-like structure. Radiation modulating device 100 may also be cylindrical in shape, pyramidal, triangular, flat and round, flat and square, flat and ovoid or any other shape or configuration suitable for completely and/or partially blocking radiation emitted from a radiation source. Furthermore, radiation modulating device 100 may have any physical dimensions suitable for blocking radiation in a given environment. For example, certain dimensions may be preferable for enhancing the attachment of radiation modulating device 100 to certain currently available radiation equipment, such as a linear accelerator. However, other embodiments of radiation modulating device 100 may have significantly larger or smaller dimensions, for example if device 100 is to be placed apart from a radiation source or directly on a target. As is evident from the foregoing description, radiation modulating device 100 may assume any of a wide variety of configurations and dimensions to enable it to block some portion of a radiation beam.

In one embodiment of the present invention, radiation modulating device 100 includes a housing 110 and a core 106. Housing includes a top 102, a bottom 104, and at least one side wall 112, and is generally configured for supporting core 106 so that core 106 may be positioned between a radiation source and a target. Core includes a plurality of segments 108, generally configured to partially or completely block one or more portions of a radiation beam. In FIG. 1, a portion of housing 110 is cut away, to better show core 106. In FIGS. 2a and 2b, a dotted line 208 and shaded region 212 designate approximately where housing 110 is cut away on FIG. 1.

In general, top 102 and bottom 104 of housing 110 are mere designations for purposes of orientation and description of radiation modulating device 100. For example, in one embodiment radiation modulating device 100 may be oriented so that top 102 is closest to a radiation source and bottom 104 is closest to a target. In another embodiment, top 102 may be closest to the target and bottom 104 may be closest to the radiation source.

Housing 110 may have any shape or size suitable for positioning core 106 between one or more given radiation sources and one or more given targets. Thus, housing 110 may be cube-shaped, box-shaped, pyramidal, cylindrical, spherical, triangular, flat, rounded or any other shape. Housing 110 may surround core 106 on several sides (as shown in FIGS. 1 and 2 where housing 110 has four side walls 112), or may only support core 106 without surrounding it. Core 106 may also be configured in any suitable shape and may either correspond or differ from housing 110 in its overall shape. For example, housing 110 may be generally box-like, while core 106 may be cylindrical. In FIGS. 1 and 2, housing 110 and core 106 core are generally box-shaped, but core 106 may be pyramidal, cylindrical, triangular, or any other shape. Thus, as is evident from the foregoing description, housing 110 may be configured in any way suitable to support and/or position core 106 and core 106 may be configured in any way suitable for completely or partially blocking one or more portions of a radiation beam.

In one embodiment, core 106 may be configured such that it is divided into two or more segments 108. Like housing 110 and core 106, segments 108 may have any shape suitable for blocking radiation in a given environment. In FIGS. 1 and 2, segments 108 have straight sides and are generally shaped as three-dimensional rectangles, but segments 108 may also be cylindrical, triangular or any other shape. According to one aspect of the present invention, one or more segments 108 may have different thickness (or “heights”) from top 102 to bottom 104 than other segments. When radiation modulating device 100 is placed between a radiation source and a target and a radiation beam passes through core 106, thicker segments 108 will block the radiation beam to a greater extent than thinner segments. Thus, to modulate a radiation beam to have different intensities across a target area, radiation modulating device 100 may be used, with segments 108 of core 106 configured to block portions of the radiation beam as desired.

In one embodiment of the present invention, with continued reference to FIGS. 1 and 2, radiation modulating device 100 may be generally box-shaped, but the measured distance across top 102 may be less than the measured distance across bottom 104. In such an embodiment, housing 110, core 106 and segments 108 may all widen from top 102 to bottom 104. The amount of widening may be determined by the amount that a radiation beam to be passed through radiation modulating device 100 will widen (referred to as “divergence” of a radiation beam). In one embodiment of the invention, for example, radiation modulating device 100 may measure between about 3 cm and about 7 cm squared on top 102, between about 4 cm and about 8 cm squared on bottom 104 and between about 5 cm and 10 cm in height (from bottom 104 to top 102). More preferably, radiation modulating device 100 may measure between about 4 cm and about 6 cm squared on top 102, between about 5 cm and about 7 cm squared on bottom 104 and between about 6 cm and about 9 cm in height. Even more preferably, radiation modulating device 100 may measure about 5.7 cm squared on top 102, about 6.6 cm squared on bottom 104 and about 8.5 cm in height.

A radiation modulating device 100 configured in this way, with a wider bottom 104 than top 102, may be placed with top 102 nearest to a radiation source and bottom 104 farthest from the radiation source. Thus, as a radiation beam passes from the radiation source through top 102 to bottom 104 and widens at the same rate that modulating device widens, radiation beam continues to move parallel to the sides of housing 110, core 106 and segments 108. Radiation modulating device 100 would thus compensate for the divergence of the radiation beam. As stated above, however, these are merely exemplary dimensions and configurations of one embodiment of radiation modulating device 100 and are not meant to limit the scope of the present invention in any way.

Housing 110 and core 106 of radiation modulating device 100 may be constructed from any material, or combination of materials, suitable for blocking radiation. Examples of such materials include, but are not limited to, metals, such as cerrobend or lead, and various ceramic materials. In one embodiment, housing 110 and core 106 may be made of the same material, such as cerrobend. However, housing 110 and core 106 may also be made of different materials. Furthermore, housing 110 and core 106 of radiation modulating device 100 may be made of any other material now known or hereafter devised.

As well as having many possible shapes, sizes and configurations, radiation modulating device 100 may be used in many different ways and for any of a number of different purposes. For example, radiation modulating device 100 may be attached to a radiation delivery device, such as a linear accelerator, and may be used for an entire radiation therapy treatment of a patient or other target. Alternatively, radiation modulating device 100 may be attached to a radiation delivery device for a portion of a treatment, and may be replaced with one or more other radiation modulating devices 100 for other portions of the treatment, either from the same or different treatment angles. Radiation modulating device 100 may also move along with a moving radiation source and/or moving target. Or radiation modulating device 100 may be placed between a stationary radiation source and target. In another embodiment, radiation modulating device 100 may be placed on the patient or other target such that a radiation beam from a radiation source passes through device 100 before entering the target. Radiation modulating device 100 may thus be incorporated into a system for radiation delivery in many different ways without altering the beneficial modulation of a radiation beam of which device 100 is capable.

As is described in further detail below, radiation modulating device 100 may be constructed by any means now known or hereafter devised by those skilled in the art for constructing such a device. For example, radiation modulating device 100 may be carved, milled or machine tooled out of one solid piece of material. Alternatively, device 100 may be comprised of multiple pieces of the same or various materials that are adhered together. In yet another embodiment, radiation modulating device 100 may be constructed from a mold. A mold may be milled, using a commonly known milling machine such as the Autimo™ 3D, manufactured by Hek Medical Technology GmbH, Lubeck, Germany. A metal, such as cerrobend, or other suitable substance may be placed in a mold to create radiation modulating device 100.

In determining how to configure radiation modulating device 100, many methods may be used. Examples below will provide more detail on one or more exemplary processes for designing and constructing radiation modulating device 100. However, it should be understood that these examples do not limit the configuration of radiation modulating device 100 or methods for making or using radiation modulating device 100 to one specific embodiment, but are merely examples.

That being said, in one embodiment of the present invention, the general configuration of a portion of radiation modulating device 100 may be based upon a given body of information. For example, in one embodiment, the overall size, shape and configuration of radiation modulating device 100 may be determined by how it will be attached to a radiation source, placed between a radiation source and a target, or placed on a target. Alternatively, the overall shape may be chosen based on other criteria, such as the workings of a machine used to build radiation modulating device 100, ease of transport of device 100 and/or other criteria. The configuration of core 106, for example, may be based on information regarding amounts of radiation that are desired to be delivered to a target. Thus, if it is desired that specific intensities of radiation arrive at some portions of a target and other intensities arrive at other portions, core 106 may be configured with segments 108 having thicknesses that correspond to those intensity specifications.

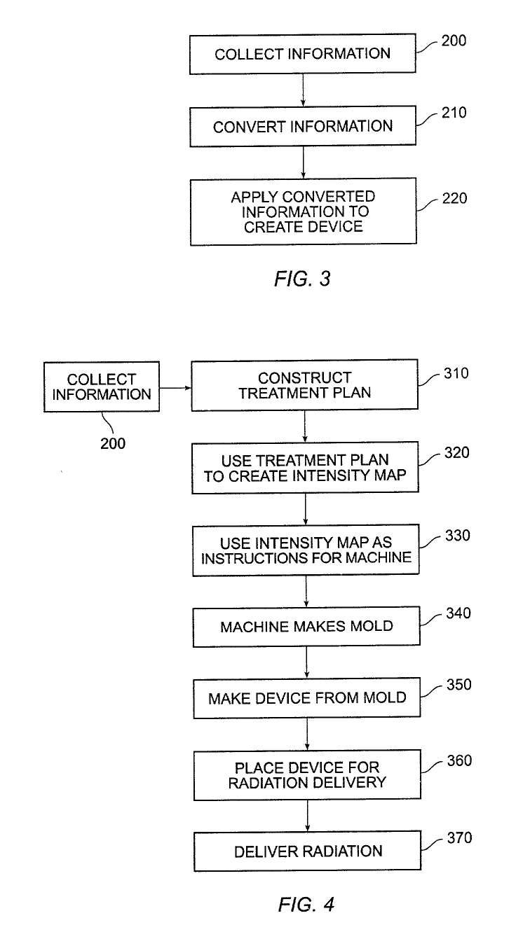

In one embodiment of the present invention, a method may be used to incorporate the above-described information into the design and construction of radiation modulating device 100. With reference to FIG. 3, such a method may generally include three basic steps—collecting information 200, converting information 210 and applying converted information to create device 220. This method is only one exemplary method, however, and the three basic steps may be altered, deleted or added to in another embodiment without departing from the scope of the present invention. For example, in another embodiment the converting step 210 may not be necessary.

Information collected (step 200) for making radiation modulating device 100 may include any suitable information. For example, one type of information may include information about the way in which radiation modulating device is to be used. For example, radiation modulating device 100 may be configured to be easily attached and detached from many currently used radiation sources, such as particle accelerators, and/or may be configured to compensate for divergence of a radiation beam, as described above. Information may also include information about a target. For example, core 106 may be configured for blocking portions of a radiation beam, based on levels of radiation that are desired to arrive at a target. Thus, information used to configure core 106 may include, for example, images of a target, such as CT scans, MRI scans, X-rays, ultrasound images, drawings on any of the aforementioned images, computer-enhanced images, conventional photographs, scanned images of photographs or any other images suitable for describing a target. Other information may include treatment parameters, which define the intensities of radiation that are desired to be delivered to various areas on a target. Additional information may include three-dimensional measurements of a target, predicted motion of a target or motion of a radiation source, characteristics of a radiation beam, densities of a target to be penetrated and/or any other information suitable for use in designing and constructing radiation modulating device 100.

When information is collected 200, or while information is being collected 200, some or all of the information may be converted 210 for use in constructing radiation modulating device 100. For example, all imaging information, treatment parameters and other data may be converted 210 into a form that may be used by one or more machines to build radiation modulating device 100. In one embodiment, a computer system may be used to convert information into a treatment plan and another computer system may be used to convert that treatment plan into a form usable a machine. In another embodiment, conversion of information 210 may be accomplished by one computer system. In yet another embodiment, information may be used to create radiation modulating device 100 without being converted.

With continued reference to FIG. 3, once information is collected (step 200) and converted (step 210), it may be applied to create radiation modulating device 100 (step 220). For example, information may be entered into a machine for building radiation modulating device 100. Radiation modulating device 100 may then be made, according to the information.

Radiation modulation device 100 may be constructed for and used in many different environments. For example, device 100 may be used in a laboratory context, to study any number of characteristics and/or effects of radiation. As such, radiation may be directed at any of a number of targets, such as laboratory animals, radiographic film or a radiation measuring device. Radiation modulating device 100 may be used in conjunction with such laboratory studies to modulate a radiation beam for purposes of testing the effects of different beam intensities or for other purposes. Many other uses for radiation modulating device may be known or devised by those skilled in the art, and the present invention is not limited to any one use or group of uses, but instead may be used for modulating radiation from any source for any reason.

That being said, in one embodiment of the present invention, radiation modulating device 100 may be constructed and used for radiation therapy of cancer patients. As described above, cancer is a costly disease, from both a healthcare and an economic perspective. Thus, technological advances in cancer therapy that provide improved therapeutic results with equal or reduced costs are extremely valuable. In accordance with one embodiment of the present invention, radiation modulating device 100 provides an affordable alternative to current IMRT systems. That is, radiation modulating device 100 in various forms may be configured to be compatible with radiation therapy systems currently in use, such as analog linear accelerators.

With reference to FIG. 4, one embodiment of a method for making and using radiation modulating device 100 may include multiple steps. Again, one or more of the steps described in FIG. 4 may be altered or deleted and/or the order of steps may be changed without departing from the scope of the present invention. FIG. 4 and the description below are provided for exemplary purposes only. That being said, a first step in one method for making and using radiation modulating device 100 may include collecting information 200. This step is described above, with reference to FIG. 3. As in FIG. 3, information may include any suitable information for making and using radiation modulating device 100.

Information may be used to create a treatment plan 310. A treatment plan may include any information and combination of information about a person or object to be treated, one or more pieces of equipment to be used for the treatment, characteristics of a radiation source to be used in the treatment and the like. A treatment plan may also include drawings or outlines on images of a target area, parameters defining amounts of radiation to be delivered to various target areas, and the like. In one embodiment of the present invention, an “inverse treatment plan” may be used. Generally, inverse treatment plans are based on information about a target and treatment parameters, such as doses of radiation that a clinician desires to be delivered to various areas on a target. This is distinguishable from treatment plans which are based on amounts of radiation to be emitted from a radiation source (rather than amounts of radiation to arrive at a target). In various other embodiments, treatment plans make take various forms and may include any suitable information either now available for generating a treatment plan or discovered in the future for generating treatment plans.

A treatment plan may next be used to make an intensity map 320. An intensity map may comprise any list or map of intensities which are desired to be delivered to a target. An “intensity” may be any suitable measurement of radiation intensity. In one embodiment, an intensity on an intensity map may represent a percentage of total intensity emitted by a radiation source. For example, if a radiation source emits a radiation beam having intensity X and it is desired that a portion of a target receive a dose that is 50% of intensity X, the “intensity” on an intensity map for that portion of the target may be represented as “50.” Furthermore, each number on an intensity map may correspond to a particular area on a target. In various other embodiments, any other suitable system for representing one or more intensities to be delivered to a target may be used.

In accordance with one embodiment of the present invention, an intensity map may be used as input for a machine configured to assist in making radiation modulating device 100 (step 330). In addition to an intensity map, such input may include any suitable information and may be designed for any suitable machine now known or hereafter discovered that may make, or assist in making, radiation modulating device. For example, input for such a machine may include, but is not limited to, dimensions, thicknesses, angles and the like which may be used with a milling machine. A milling machine may be used to create radiation modulating device 100 from a piece of material or may be used to create a mold of radiation modulating device 100. In accordance with various aspects of the invention, any other suitable input for any suitable machine may be included in step 330 without departing from the scope of the present invention.

With continued reference to FIG. 4, the next step in one embodiment of a method for making radiation modulating device 100 may be for a machine to make a mold of device 100. A mold may be made of any suitable material and may assume any of a variety of configurations. For example, molds may be made of high-density styrofoam, wood or any other suitable material for making a mold. Furthermore, one piece of material may be used to make one mold for making one radiation modulating device 100 or one piece of material may be used to make multiple molds for making multiple radiation modulating devices 100. For example, two, five, ten or more molds may be milled into one block of high-density styrofoam. Finally, in various other embodiments, radiation modulating device 100 may be made without the use of a mold.

Next, radiation modulating device 100 may be made from a mold (step 350). This step may be accomplished by any of a variety of different materials and methods. In one embodiment, for example, a molten or liquid substance may be poured into a mold and allowed to harden to form device 100. In another embodiment, crystalline material may be placed in a mold to form device 100. In fact, any suitable material may be used with any suitable mold to make radiation modulating device 100.

Once made, radiation modulating device 100 may be placed in a location for use 360. For example, device 100 may be configured to facilitate attachment to a linear accelerator by any of a variety of apparatus and methods. In one embodiment, radiation modulating device 100 may be configured to be attached to a tray or platform which may then be attached to a linear accelerator. In another embodiment, radiation modulating device 100 may be placed on a stand or other stationary object that is separate from a linear accelerator and radiation beam may be aimed to pass through device 100. In yet another embodiment, radiation modulating device may be placed on an apparatus configured to move in conjunction with movements of a linear accelerator, for example to deliver radiation to a target from multiple angles. Any other suitable apparatus or method may be used, in various embodiments, to place device 100 for use.

Finally, once radiation modulating device 100 is placed for use, radiation may be delivered (step 370). A radiation delivery device, such as a linear particle accelerator may be activated, for example, to emit a radiation beam. The radiation beam may then travel through radiation modulating device 100 and arrive at a target. Radiation delivery 370 may continue for any suitable time, depending on objectives of the delivery. Radiation delivery 370 may also be accomplished from multiple angles. In one embodiment, a different radiation modulating device 100 may be used for each angle of radiation delivery. Again, any suitable apparatus and method for delivering radiation may be used without departing from the scope of the present invention.

In accordance with various aspects of the present invention, various computer software elements may be included. For example software elements may be used to create intensity maps from inverse treatment plans or device thickness maps from intensity maps. Software elements included in various embodiments of the present invention may be implemented with any programming or scripting language, such as C+, C++, Java, COBOL, assembler, PERL, eXtensible Markup Language (XML), and the like, with the various algorithms being implemented with any combination of data structures, objects, processes, routines or other programming elements. further, it should be noted that the present invention may employ any number of conventional techniques for data transmission, signaling, data processing, network control, and the like. Still further, the invention could be used to detect or prevent security issutes, with a client-side scripting language, such as JavaScript, VBScript, or the like. for a basic introduction to cryptography, please review “Applied Cryptography: Protocols, Algorithms, and Source Code in C,” by Bruce Scheider, published by John Wiley & Sons (second edition, 1996), which is hereby incorporated by reference.

Various embodiments of the present invention may further include one or more systems for transferring and/or storing information. In one embodiment, for example, inverse treatment plans, intensity maps, device thickness maps, and the like may be transferred between multiple locations, such as a clinic and a facility for making radiation modulating device 100. Communication between one or more clinicians, manufacturers of radiation modulating devices 100 and the like may be accomplished through any suitable communication means, such as, for example, a telephone network, Intranet, Internet, online communications, off-line communications, wireless communications, and/or the like. For security reasons, any databases, systems, or components of the present invention may consist of any combination of databases or components at a single location or at multiple locations, wherein each database or system includes any of various suitable security features, such as firewalls, access codes, encryption, de-encryption, compression, decompression, and/or the like.

In accordance with various aspects of the present invention, systems or methods for making radiation modulating device 100 may include a host server or other computing systems, including a processor for processing digital data, a memory coupled to said processor for storing digital data, an input digitizer coupled to the processor for inputting digital data, an application program stored in said memory and accessible by said processor for directing processing of digital data by said processor, a display coupled to the processor and memory for displaying information derived from digital data processed by said processor and a plurality of databases. Databases may be configured to store, for example, inverse treatment planning data, intensity map data, device thickness map data and the like. A database may be any type of database, such as relational, hierarchical, object-oriented, and/or the like. Common database products that may be used to implement database include DB2 by IBM (White Plains, N.Y.), any of the database products available from Oracle Corporation (Redwood Shores, Calif.), Microsoft Access by Microsoft Corporation (Redmond, Wash.), or any other database product. A database may be organized in any suitable manner, including as data tables or lookup tables.

A clinician may access and use a system for making radiation modulating device 100 by means of a computer, including an operating system (e.g., Windows 95/97/9812000, Linux, Solaris, etc.) as well as various conventional support software and drivers typically associated with computers. In an exemplary embodiment, a clinician may send instructions, intensity maps, treatment plans and the like via a network to a third party that creates radiation modulating device 100. Thus, the clinician's computer can be in a home or business environment with access to a network and may have access to the third party via the Internet through a commercially-available web-browser software package.

As described above, many exemplary embodiments of apparatus and methods are contemplated by the present invention. Therefore, the following example is not meant to be limiting in any way, but is provided for further understanding and example only.

That being said, using one embodiment of the invention, radiation modulating device 100 was be used to enhance the treatment of a patient with prostate cancer. First, CT scans of the region of a patient's body containing the prostate were acquired. As the patient's prostate was to be treated with radiation from five different angles, CT scans from the five prospective treatment angles were acquired. Using the CORVUS system, an inverse treatment plan was created. CORVUS is a commonly available inverse treatment planning system provided by NOMOS Corporation (Sewickley, Pa.). Further information about the CORVUS system can be found on NOMOS' web site, at www.nomos.com, the entire contents of which is hereby incorporated by reference.

To create the inverse treatment plan with CORVUS, various regions in and around the prostate were outlined and treatment parameters were defined. Treatment parameters included minimum doses of radiation which would be sufficient for delivery to certain outlined target areas (such as central areas of prostate tumor) and maximal doses which would be acceptable for delivery to other outlined target areas (such as vital, healthy tissues surrounding the prosteate). In other words, lower dose limits were set for target areas where the most radiation was desired and upper dose limits were set for normal structures where no radiation was desired.

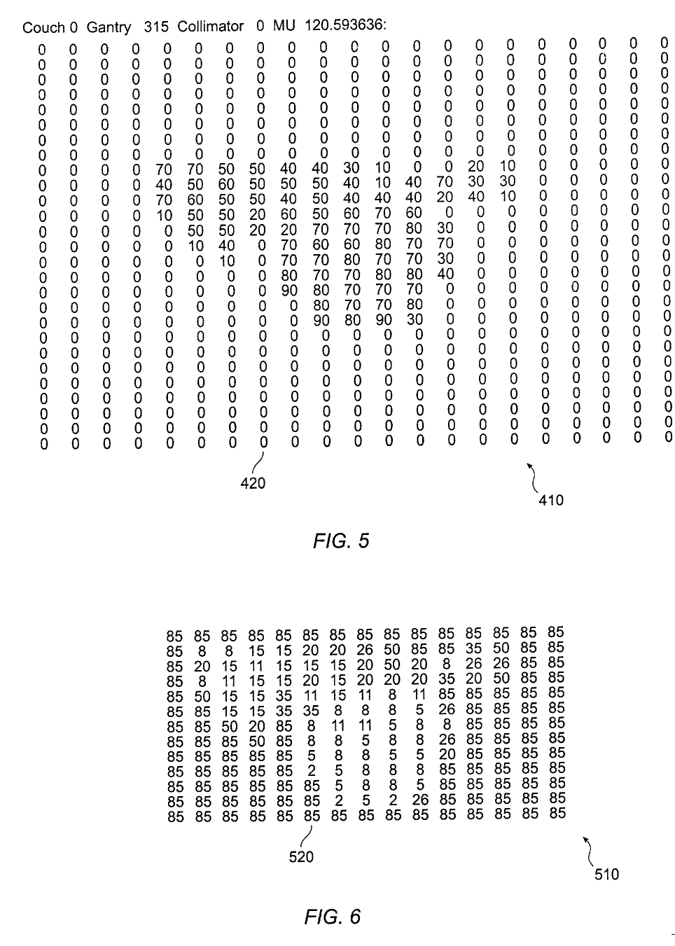

After information was entered into the CORVUS system, CORVUS produced an intensity map, such as the one shown in FIG. 5. In FIG. 5, each number 420 on intensity map 410 corresponds to a “cell” or area of a target. A cell may represent any size or shape of an area on a target. In this example, each cell represented a target area of approximately 1 cm squared. Each number 420 on intensity map 410 thus represented a percentage of the maximum strength of a radiation beam which would be delivered to the cell corresponding to that number 420. The beam in this example was measured in monitor units, so the percentage number shown in the intensity map is a percentage of the maximum monitor units. A “0” on intensity map 410 means that the radiation beam was to be blocked entirely in that cell, so that as close to 0% as possible of the beam would arrive at that cell on the target. An “80” on intensity map 410 means that the radiation beam was to be blocked only 20% in that cell and that 80% of the radiation beam was to be allowed to arrive at that cell on the target. Typically, an intensity map 410 may be made for each gantry angle from which a patient will be treated, and that was done in this example, producing five intensity maps.

In this example, the intensity map, such as in FIG. 5, was used to make a device thickness map, as shown in FIG. 6. Device thickness map 510 may be thought of as an approximate inverse of intensity map 410. In this example, each number 520 on device thickness map 510 represents a thickness that a corresponding segment 108 of radiation modulating device 100 would have, in order to sufficiently block a radiation beam in the corresponding cell. In this example, the thicknesses were measured in millimeters, though other measurements may be used. For example, to block approximately 100% of a particular beam, allowing approximately 0% to arrive at a target, a particular cell of radiation modulating device 100 was designed to be 85 mm thick. The thickness for each x and y location (or cell) on device thickness map 510 was calculated using commercially available mathematical software and experimental data, along with the following equation:

(t)x,y=(Ln(% transmission)x,y)/μ

where “t” is the thickness of a segment 108 corresponding to a particular cell, “x,y” are the coordinates of the particular cell, “Ln” is the natural logarithm operator, “% transmission” is the number for that particular cell from the intensity map, and “μ” is the linear attenuation coefficient of the material from which radiation modulating device 100 was to be made (cerrobend in this example).

Next, device thickness maps were used as instructions for a milling machine. A commercially available milling machine, the Autimo™ 3D from Hek Medical Technology GmbH, Germany, was adapted to interpret device thickness maps and to mill molds, or negative impressions, of radiation modulating devices 100. (Again, in various embodiments of the invention, device thickness maps, intensity maps and the like may not be required. Instead, for example, one or more types of raw data may be entered into a machine in one embodiment). To manufacture a radiation modulating device 100 like the one depicted in FIGS. 1 and 2, with widening dimensions from top 102 to bottom 104, adaptations or adjustments of the commercially available milling machine were made. These adaptations enabled the machine to operate with four degrees of freedom, which enabled the machine to create a mold configured to incorporate the widening dimensions of radiation modulating device 100 shown in FIGS. 1 and 2. The adapted milling machine was then used to mill five molds into five pieces of high-density styrofoam.

Once the molds were made, molten cerrobend was used to fill each mold and was then allowed to harden to an extent sufficient to allow the cerrobend to be removed from each mold in one piece. The hardened cerrobend was then removed from the molds, rendering five radiation modulating devices 100. In this example, radiation modulating devices 100 were configured similar to the configurations shown in FIGS. 1 and 2. Generally, the devices 100 were block-like. However, the distance across top 102 was less than the distance across bottom 104 and segments 108 and housing 110 widened from top 102 to bottom 104. Furthermore, each segment 108 was configured to block a particular portion of a radiation beam, to achieve a desired distribution of radiation at the target area on the patient. As such, segments 108 generally had discrete, box-shaped configurations with approximately square or rectangular tops and bottoms and four side walls. Radiation modulating devices 100 made in this example had outer dimensions of about 5.7 cm squared on top 102, about 6.6 cm squared on bottom 104 and about 8.5 cm in height.

Each radiation modulating device 100 was then tested, to compare its functionality to that of a currently available IMRT system. Such testing involved attaching one radiation modulating device 100 to a linear accelerator via a commonly available tray (as used with conventional compensators), placing a piece of film where a target area on a patient would reside during a treatment, and activating the linear accelerator. The radiation beam emitted from the accelerator, which was then partially blocked by radiation modulating device 100, developed a portion of the radiographic film. Sections of the film receiving the most radiation were developed most and sections receiving the least radiation were developed least. Using radiographic test film to verify the intensities of radiation being delivered to a target is well known to those skilled in the art and, in fact, is the preferred method for testing current IMRT systems. Thus, the results of radiation modulation using radiation modulating device 100 were compared to results of current IMRT systems using this radiographic test film technique. At each of five gantry angles, one radiation modulating device 100 was compared to an IMRT system, using the same treatment parameters for both, as if the piece of film were the same patient target area.

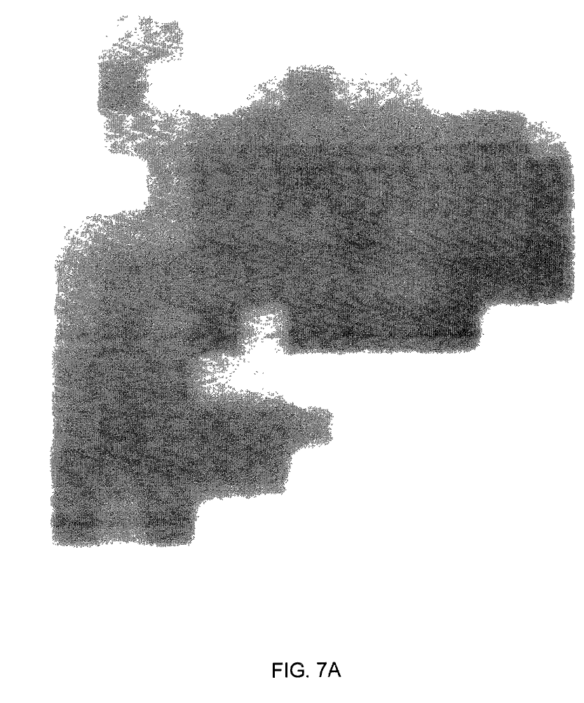

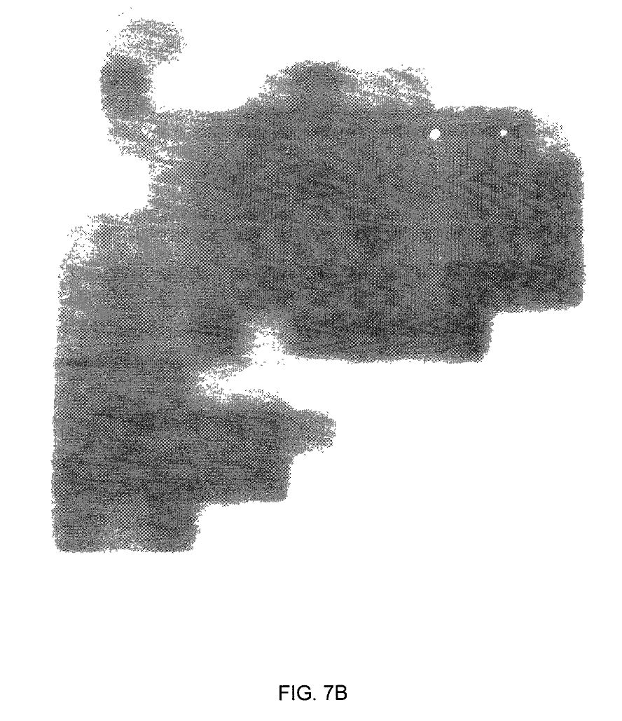

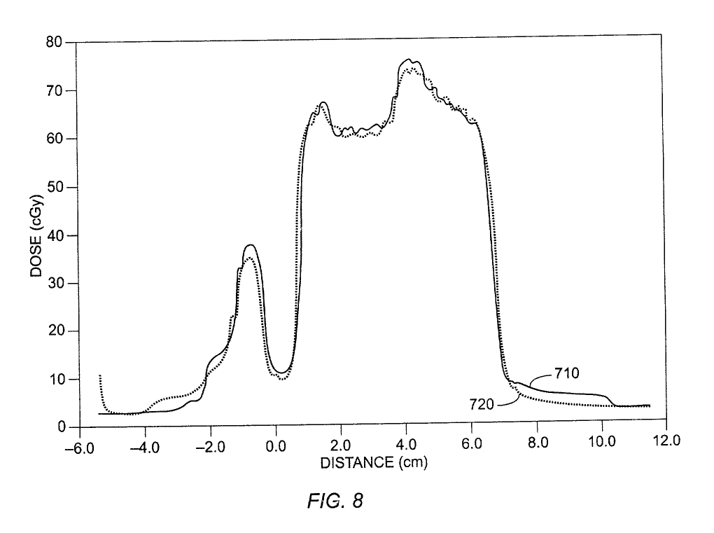

Copies of pieces of test film developed in this way are depicted in FIG. 7a, which shows a test film taken using one radiation modulating device 100, and FIG. 7b, which shows a test film taken using the same target and treatment parameters and the same linear accelerator, but using a currently available IMRT system. The tests showed that radiation modulating device 100 and the IMRT system produced almost identical configurations of radiation at the target. FIG. 8 is a graph, showing a delivered dose calculated for a sampling of cells (i.e., 1 cm square areas on the target) taken from the central, horizontal line drawn across a test film such as those in FIGS. 7a and 7b. On the graph, the light-colored line 710 represents the radiation dose intensities arriving at cells of the test film after the radiation had passed through radiation modulating device 100. The dark-colored line 720 represents the radiation dose intensities arriving at cells of the test film after the same radiation beam had passed through a currently available MLC of an IMRT system. The slight differences between these two lines were found to be insignificant, thus demonstrating that radiation modulating device 100 is at least as effective as IMRT in modulating radiation to create a desired three-dimensional distribution of a radiation dose at a target.

The present invention has been described above with reference to various exemplary embodiments. However, those skilled in the art will recognize that changes and modifications may be made to various exemplary embodiments without departing from the scope of the present invention. For example, radiation modulating device 100 may assume any of a multitude of configurations and housing 110 and core 106 of radiation modulating device 100 may assume any configurations suitable for wholly or partially blocking one or more portions of a radiation beam. Furthermore, examples of methods for making and using radiation modulating device 100 which were described above may be altered in many ways without departing from the scope of the present invention. Steps for making device 100 may be eliminated, their order may be changed, or various elements of the steps may be altered in various embodiments. For example, in one embodiment, no thickness map may be made. In another embodiment, a machine other than a milling machine may be used and/or device 100 may be made without use of a mold. Thus, radiation modulating device 100 may assume any suitable configuration, may be manufactured by many various methods and may be used for a variety of purposes. These and other changes or modifications are intended to be included within the scope of the present invention as set forth in the appended claims.

We claim:

1. A system for delivering radiation therapy to a cancer patient, comprising:
a radiation delivery device;
a radiation modulating device; and
a target.

2. The system of claim 1, wherein said radiation delivery device comprises a linear accelerator.

3. The system of claim 1, wherein said radiation modulating device comprises:
a core having at least two segments; and
a housing having a top, a bottom, and at least one side wall, said housing configured to enable the positioning of said core between said radiation delivery device and said target,
wherein at least one of said segments is configured to partially block portions of a radiation beam from arriving at said target, and
wherein at least one of said segments is configured to permit portions of a radiation beam to arrive at said target.

4. The system of claim 3, wherein said radiation modulating device has a block-like configuration.

5. The system of claim 3, wherein the length measured across the center of said top is smaller than the length measured across the center of said bottom.

6. The system of claim 3, wherein said at least one side wall and said at least two segments have wider dimensions towards said bottom than towards said top.

7. The system of claim 6, wherein said wider dimensions are based on the divergence of a radiation beam emitted from said radiation delivery device.

8. The system of claim 1, wherein said target includes an area of a cancer patient's body.

9. A radiation modulating device, comprising:
a core having at least two segments; and
a housing having a top, a bottom, and at least one side wall, said housing configured to enable the positioning of said core between a radiation delivery device and a target,
wherein at least one of said segments is configured to partially block portions of a radiation beam from arriving at said target, and
wherein at least one of said segments is configured to permit portions of a radiation beam to arrive at said target.

10. The device of claim 9, wherein said radiation modulating device has a block-like configuration.

11. The device of claim 9, wherein said radiation modulating device is comprised of metal.

12. The device of claim 11, wherein said metal comprises at least one of cerrobend and lead.

13. The system of claim 9, wherein the length measured across the center of said top is smaller than the length measured across the center of said bottom.

14. The system of claim 9, wherein said at least one side wall and said at least two segments have wider dimensions towards said bottom than towards said top.

15. The system of claim 9, wherein said wider dimensions are based on the divergence of a radiation beam emitted from said radiation delivery device.

16. A method of making a radiation modulating device, comprising:
acquiring information;
using said information to create a distribution of desired radiation to be delivered to a target; and
using said distribution of desired radiation to make said radiation modulating device.

17. The method of claim 16, further comprising:
acquiring one or more images of said target;
outlining one or more areas on each of said one or more images; and
defining a minimum or a maximum amount of radiation to be delivered to each of said one or more areas.

18. The method of claim 16, further comprising:
using said distribution of desired radiation as instructions for a milling machine;
making a mold with said milling machine, based on said distribution of desired radiation; and
using said mold to make said radiation modulating device.

19. The method of claim 16, wherein said information includes at least one of a CT scan, an MRI scan, a conventional radiograph, a sonogram and a digital photograph of said target.

20. The method of claim 16, wherein said information includes at least one of a minimum dose of radiation to be delivered to an area of said target and a maximum dose of radiation to be delivered to said area of said target.

21. The method of claim 16, wherein said using said information to create a distribution of desired radiation includes creating an intensity map, said intensity map comprising an identification of at least two different levels of intensity of radiation to be delivered to said target.

22. The method of claim 16, wherein said using said distribution of desired radiation to make said radiation modulating device includes creating a thickness map, said thickness map comprising an identification of at least two different thicknesses for two different segments of said radiation modulating device.
